SEQUENCE LISTING (2 sequences)

SEQ ID NO: 1 - DNA, 813 bp, Homo sapiens
atggagaaccagcctgtgaggtggcgggccctgccaggcctcccacgccctcctgggctccccgcagccccctggctcctccttggcgtgctgctgctgcccgggaccctgcgactggcaggaggacagtcagtgacccacaccggcctgcccatcatggcctccctggccaacacagctatctccttcagctgcaggatcacctatccatacactccccaattcaaggttttcacagtcagctactttcatgaagatctccagggacagaggagccctaagaagccaacaaactgccaccctggactgggcacagagaaccagagccacaccctggactgccaggtcacccttgtgctgccgggagcatcggccactggcacctactactgctctgtccactggccacactccacggtgagaggcagcggcaccttcatcctggtcagagacgcagggtaccgagagcccccgcagagtccacagaagctcctgctctttggcttcaccggcctcctgagtgtcctgagtgtagtgggcacggccctgctgctctggaacaagaagcggatgcggggtccagggaaggaccccaccaggaagtgcccagatccaagatctgccagcagccccaagcagcatccttcagaatctgtctacacagctctgcagcgccgcgagaccgaggtctatgcctgcatcgagaatgaggatggcagctcacccaccgccaagcagagccccctctcccaggagagaccgcatagattcgaagatgatggcgaacttaacctggtctatgaaaatctctag

SEQ ID NO: 2 - protein, 270 aa, Homo sapiens
MENQPVRWRALPGLPRPPGLPAAPWLLLGVLLLPGTLRLAGGQSVTHTGLPIMASLANTAISFSCRITYPYTPQFKVFTVSYFHEDLQGQRSPKKPTNCHPGLGTENQSHTLDCQVTLVLPGASATGTYYCSVHWPHSTVRGSGTFILVRDAGYREPPQSPQKLLLFGFTGLLSVLSVVGTALLLWNKKRMRGPGKDPTRKCPDPRSASSPKQHPSESVYTALQRRETEVYACIENEDGSSPTAKQSPLSQERPHRFEDDGELNLVYENL

(19) United States
(12) Patent Application Publication
(10) Pub. No.: US 2009/0022710 A1
(43) Pub. Date: Jan. 22, 2009

(54) NUCLEAR FACTOR OF ACTIVATED T CELLS RECEPTOR

(75) Inventors: Zhengbin Yao, Sugar Land, TX (US); Guanghu Hu, Houston, TX (US); Kang Li, Missouri City, TX (US); Jianhua Yang, Pearland, TX (US)

Correspondence Address: Nikolaos C. George;JONES DAY, 222 E. 41st. Street, New York, NY, 10017-6702, US

(73) Assignee: Genentech, Inc.

(21) Appl. No.: 12/016,447

(22) Filed: Jan. 18, 2008

Related U.S. Application Data
(62) Division of application No. 10/528,326, filed on Mar. 7, 2006, now abandoned.
(60) Provisional application No. 60/412,157, filed on Sep. 19, 2002.

(51) Int. Cl. A61K 39/395; G01N 33/53; C07K 14/00; C07H 21/00; A61P 37/02; C12N 5/16; C12P 21/00; C12N 15/64

(52) U.S. Cl. 424/130.1; 435/7.24; 530/350; 536/23.1; 435/358; 435/69.1; 435/320.1

(57) ABSTRACT

The present invention provides a novel transmembrane protein, which is a nuclear factor of activated T cells (‘NFAT’) receptor, and related compositions and methods.

### BACKGROUND OF THE INVENTION

1. Field of the Invention

This invention relates generally to cellular receptors and particularly to nuclear factor of activated T cells (“NFAT”) activating receptors.

2. Description of lie Prior Art

Activated T lymphocytes secrete cytokines that regulate the activity of the immune system and enable the immune system to develop an effective immune response. The regulation of cytokine production can occur at the level of transcription initiation of the cytokine gene. A family of protein transcriptional factors designated “nuclear factor of activated T cells” (NFAT) plays a critical role in the transcriptional regulation of cytokine genes. NFAT proteins are known to play a key role in the regulation of transcription of a wide variety of cytokines and cell surface receptors that mediated important immune functions, e.g., mterleukin-2, interleukin-4, interleukin-5, interleukin-13, interferon-γ, tumor necrosis factor-α, GM-CSF, CD40L, and CTLA-4. The best known and well characterized members for fee NFAT family are NFAT1, NFAT2, NFAT3, and NFAT4.

NFAT protein activation is regulated through a process mat involves NFAT protein dephosphorylation, nuclear translocation, and DNA binding. In resting cells, phosphorylated NFAT proteins reside in the cytoplasm and have a low binding affinity for DNA. Various stimuli that trigger calcium mobilization cause the rapid dephosphorylation of NFAT proteins through a process mediated by the Ca2+/calmodulin-dependent protein phosphatase calcineurin. Dephosphorylated NFAT proteins with an exposed nuclear localization signal translocate into the nucleus where they bind to DNA with increased affinity and mediate target gene transcription. NFAT proteins are expressed not only in T cells but also in a diverse group of immune and non-immune cell types. NFAT proteins have been implicated in the activation of mast cells, B lymphocytes, and NK cells. In these cells, NFAT proteins are activated by stimulating receptors coupled to calcium/calcineurin signals, e.g., the antigen receptors on T and B cells, Fcε receptors on mast cells and basophils, the Fcγ receptors on macrophages and NK cells, and receptors coupled to heterotrimeric G proteins.

The following patents disclose polypeptides mat are associated with the transcription complex NFAT and associated polynucleotides, antibodies, and related methods and products. U.S. Pat. No. 5,837,840 issued to Crabtree, et al. on Nov. 17, 1998 (assigned to Board of Trustees of Leland Stanford Jr. University (Stanford, Calif.)) entitled “NF-AT polypeptides and polynucleotides”; U.S. Pat. No. 6,096,515 issued to Crabtree, et al. on Aug. 1, 2000 (assigned to Board of Trustees of the Leland Stanford Junior University (Stanford, Calif.)) entitled “NF-AT polynucleotides”; U.S. Pat. No. 6,150,099 issued to Crabtree, et al. on Nov. 21, 2000 (assigned to Board of Trustees of the Leland Stanford Junior University (Stanford, Calif.)) entitled “NF-AT polypeptides and polynucleotides”; U.S. Pat. No. 6,171,781 issued to Crabtree, et al. on Jan. 9, 2001 (assigned to The Board of Trustees of the Leland Stanford Junior University (Stanford, Calif.)) entitled “NF-AT polypeptides and polynucleotides”; U.S. Pat. No. 6,197,925 issued to Crabtree, et al. on Mar. 6, 2001 (assigned to Sara Lee Corporation (Winston-Salem, NQ) entitled “NF-AT polypeptides and polynucleotides”; U.S. Pat. No. 6,312,899 issued to Crabtree, et al. on Nov. 6,2001 (assigned to Board of Trustees of the Leland Stanford Junior University (Palo Alto, Calif.)) entitled “NF-AT polypeptides and polynucleotides”; U.S. Pat. No. 6,352,830 issued to Crabtree, et al. on Mar. 5,2002 (assigned to The Board of Trustees of the Leland Stanford Junior University (Stanford, Calif.)) entitled “NF-AT polypeptides and polynucleotides and screening methods for immunosuppressive agents”; and U.S. Pat. No. 6,388,052 issued to Crabtree, et al. on May 14, 2002 (assigned to Board of Trustees of the Leland Stanford Junior University (Stanford, Calif.)) entitled “NF-AT polypeptides and polynucleotides.”

Although much is known about NFAT proteins and the mechanism by which they affect cytokine production, there exists a continuing need to understand the NFAT pathway and to use this understanding to develop compositions and methods useful to modulate the pathway, including agonists and antagonists such as antibodies that regulate cytokine and cell surface receptor expression and screening methods that are useful to identify drugs that prevent or treat cytokine and receptor related disease.

### SUMMARY OF THE INVENTION

It is, therefore, an object of the invention to provide novel NFAT activating receptors capable of interacting with nuclear factor of activated T cells ligand proteins.

It is another object of the invention to provide agonists or antagonists that bind to native NFAT activating receptors and inhibit or activate the expression or action of such receptors.

It is another object of the invention to provide antibodies that bind to NFAT activating receptors and methods for producing such antibodies.

It is further object of the invention to provide nucleotide sequences that encode novel NFAT activating receptors capable of interacting with nuclear factor of activated T cells ligand proteins.

It is another object of the invention to provide vectors comprising nucleotide sequences that encode novel NFAT activating receptors and host cells containing such vectors.

It is a further object of the invention to provide a screening method for identifying NFAT activating receptor agonists and antagonists and for determining whether pharmaceuticals are likely to cause undesirable side effects when administered to an animal.

It is another object of the present invention to provide a method for blocking or modulating the expression of a NFAT activating receptor.

It is another object of the present invention to provide a method for diagnosing the predisposition of a patient to develop diseases caused by the unregulated expression of cytokines.

It is a farmer object of the invention to provide a method for preventing or treating NFAT protein mediated diseases in a mammal.

It is another object of the present invention to provide a diagnostic method for detecting NFAT activating receptors expressed in specific cells, tissues, or body fluids.

It is another object of the present invention to provide a method for isolating and purifying NFAT activating receptors from recombinant cell culture, contaminants, and native environments.

It is another object of the present invention to provide a method for inducing tolerance in a mammal that may experience an unwanted immune response.

These and other objects are achieved by providing a novel nuclear factor of activated T cells (“NFAT”) activating receptor having the amino sequence shown in SEQ ID NO:1, the nucleotide sequence that code for the receptor, and the vectors and host cells that express nucleotide sequence and produce the receptor. The receptor is used to produce agonist and antagonist antibodies useful for affecting the cellular production of cytokines and cellular receptors and ligands. The antibodies are useful for screening for receptor agonists and antagonists and for screening pharmaceuticals to determine if they are likely to cause undesirable side effects when administered to an animal for medicinal purposes.

Other and further objects, features, and advantages of the present invention will be readily apparent to those skilled in the art.

### DETAILED DESCRIPTION OF THE INVENTION

Definitions

The term “purified polypeptide” means a polypeptide identified and separated from at least one contaminant polypeptide ordinarily associated with the purified polypeptide in its native environment, particularly a polypeptide separated from its cellular environment.

The term “isolated polynucleotide” means a polynucleotide identified and separated from at least one contaminant polynucleotide ordinarily associated with the isolated polynucleotide in its native environment, particularly a polynucleotide separated from its cellular environment.

The term “native” when used to describe a polynucleotide, polypeptide sequence, or other molecule means a polypeptide, polynucleotide, or other molecule as found in nature, e.g., a polypeptide or polynucleotide sequence mat is present in an organism such as a virus or prokaryotic or eukaryotic cell that can be isolated from a source in nature and that has not been intentionally modified to change is structure, properties, or function. An unisolated cellular polynucleotide having the nucleotide sequence shown in SEQ ID NO:1 is a native polynucleotide and unpurified cellular polypeptide having the amino acid sequence shown in SEQ ID NO:2 is a native polypeptide.

The term “percent sequence identity” means the percentage of sequence similarity found in a comparison of two or more nucleotide or amino acid sequences. Percent identity can be determined electronically, e.g., by using the MEGALIGN program (DNASTAR, Inc., Madison Wis.). The MEGALIGN program creates alignments between two or more sequences according to different methods, e.g., the clustal method. (See, e.g., Higgins, D. G. and P. M. Sharp (1988) Gene 73:237-244.) The clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no similarity between the two amino acid sequences are not included in determining percentage similarity. Percent identity between nucleotide sequences is counted or calculated by methods known in the art, e.g., the Jotun Hein method given in Hein, J. (1990) Methods Enzymol. 183:626-645. Identity between sequences can also be determined by other methods known in the art, e.g., by varying hybridization conditions.

The term “variant” when used to describe a polynucleotide sequence means a nucleotide sequence that differs from its native counterpart by one or more nucleotides and either has the same or similar biological function as its native counterpart or does not have the same or similar biological function as its native counterpart but is useful as a probe to identify or isolate its native counterpart. Preferred variants are nucleotide sequences having at least 85 percent sequence identity when compared to its native counterpart, preferably at least 90 to 95 percent sequence identity, and most preferably at least 99 percent sequence identity, and nucleotide sequences that bind to native sequences or their complement under stringent conditions. Most Preferred variants are nucleotide sequences that code for the same amino acid sequence as its native counterpart but differ from the native nucleotide sequence based only on the degeneracy of the genetic code.

The term “variant” when used to describe a polypeptide sequence means an amino acid sequence that differs from its native counterpart by one or more amino acids, including modifications, substitutions, insertions, and deletions, and either has the same or similar biological function as its native counterpart or does not have the same or similar biological function as its native counterpart but is useful as an immunogen to produce antibodies that bind to its native counterpart or as an agonist or antagonist for its native counterpart Preferred variants are polypeptides having at least 70 percent sequence identity when compared to its native counterpart, preferably at least 85 percent sequence identity, and most preferably at least 95 percent sequence identity. Most Preferred variants are polypeptides with conservative amino acid substitutions.

The term “fragment” when used to describe a polynucleotide means a nucleotide sequence subset of its native counterpart that binds to its native counterpart or its complement under stringent conditions. Preferred fragments have a nucleotide sequence of at least 25 to 50 consecutive nucleotides of the native sequence. Most preferred fragments have an amino acid sequence of at least 50 to 100 consecutive nucleotides of the native sequence.

The term “fragment” when used to describe a polypeptide means an amino acid sequence subset of its native counterpart that either retains any biological activity of its native counterpart or acts as an immunogen capable of producing an antibody that binds to its native counterpart. Preferred fragments have an amino acid sequence of at least 10 to 20 consecutive amino acids of the native sequence. Most preferred fragments have an amino acid sequence of at least 20 to 30 consecutive amino acids of the native sequence.

The term “agonist” means any molecule that promotes, enhances, or stimulates the normal function of the NFAT activating receptor. One type of agonist is a molecule that interacts with the NFAT activating receptor in a way that mimics its ligand, including an antibody or antibody fragment

The term “antagonist” means any molecule that blocks, prevents, inhibits, or neutralizes the normal function of the NFAT activating receptor. One type of antagonist is a molecule that interferes with the interaction between NFAT activating receptor and its ligand, including an antibody or antibody fragment Another type of antagonist is an antisense nucleotide that inhibits proper transcription of native NFAT activating receptor.

The term “conservative amino acid substitution” means that an amino acid in a polypeptide has been substituted for with an amino acid having a similar side chain. For example, glycine, alanine, valine, leucine, and isoleucine have aliphatic side chains; serine and threonine have aliphatic-hydroxyl side chains; asparagine and glutamine have amide-containing side chains; phenylalanine, tyrosine, and tryptophan have aromatic side chains; lysine, arginine, and histidine have basic side chains; and cysteine and methionine have sulfur-containing side chains. Preferred conservative amino acids substitutions are valine-leucine-isoleucine, phenylalanine-tyrosine, lysine-arginine, alanine-valine, and asparagine-glutamine.

The term “stringent conditions” means (1) hybridization in 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C., (2) hybridization in 50% formamide, 5× SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C.; with washes at 42° C. in 0.2× SSC and 0.1% SDS or washes with 0.015 M NaCl, 0.0015 M sodium citrate, 0.1% Na₂SO₄ at 50° C. or similar procedures employing similar low ionic strength and high temperature washing agents and similar denaturing agents.

The term “antisense” as used herein, refers to any composition containing nucleotide sequences which are complementary to a specific DNA or RNA sequence. The term “antisense strand” is used in reference to a nucleic acid strand that is complementary to the “sense” strand. Antisense molecules include peptide nucleic acids and may be produced by any method including synthesis or transcription. Once introduced into a cell, the complementary nucleotides combine with natural sequences produced by the cell to form duplexes and block either transcription or translation. The designation “negative” is sometimes used in reference to the antisense strand, and “positive” is sometimes used in reference to the sense strand.

The term “knockout” refers to partial or complete reduction of the expression of at least a portion of a polypeptide encoded by an endogenous gene (such as the NFAT activating receptor) of a single cell, selected cells, or all of the cells of a mammal. The mammal may be a “heterozygous knockout” having one allele of the endogenous gene disrupted or “homozygous knockout” having both alleles of the endogenous gene disrupted.

This invention is not limited to the particular methodology, protocols, cell lines, vectors, and reagents described herein because they may vary. Further, the terminology used herein is for the purpose of describing particular embodiments only and is not intended to limit the scope of the present invention. As used herein and in the appended claims, the singular forms “a,” “an,” and “the” include plural reference unless the context clearly dictates otherwise, e.g., reference to “a host cell” includes a plurality of such host cells.

Because of the degeneracy of the genetic code, a multitude of nucleotide sequences encoding the NFAT activating polypeptides of the present invention may be produced. Some of these sequences will be highly homologous and some will be minimally homologous to the nucleotide sequences of any known and naturally occurring nucleotide sequence. The present invention contemplates each and every possible variation of nucleotide sequence that could be made by selecting combinations based on possible codon choices. These combinations are made in accordance with the standard triplet genetic code as applied to the nucleotide sequence that codes for naturally occurring NFAT activating receptor and all such variations are to be considered as being specifically disclosed.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the preferred methods, devices, and materials are described herein.

All patents and publications mentioned herein are incorporated herein by reference to the extent allowed by law for the purpose of describing and disclosing the proteins, enzymes, vectors, host cells, and methodologies reported therein that might be used with the present invention. However, nothing herein is to be construed as an admission that the invention is not entitled to antedate such disclosure by virtue of prior invention.

### THE INVENTION

### Polypeptides

In one aspect, the present invention provides a purified polypeptide comprising an amino sequence selected from the group consisting of SEQ ID NO:2; a variant of SEQ ID NO:2; a fragment of SEQ ID NO:2; an amino acid sequence encoded by an isolated polynucleotide comprising a nucleotide sequence selected from the group consisting of SEQ ID NO: 1; a variant of SEQ ID NO: 1; and a fragment of SEQ ID NO: 1.

The purified polypeptides of the present invention are preferably NFAT activating receptors involved in the transcriptional regulation of various cytokine and cellular receptor genes. The receptors are preferentially expressed in human immune related cells and tissues, especially neutrophils, monocytes, lymphocytes, mast cells, spleen tissue, and lung tissue. The receptors are used to create antibodies that bind to the receptors and influence receptor structure, properties, or function, including biological function. Preferably, the antibodies function as receptor agonists to activate the production of cytokines and cellular receptors or as receptor antagonists to inhibit the production of cytokines and cellular receptors.

### Agonists and Antagonists

In another aspect, the present invention provides agonists and antagonists that specifically bind to NFAT activating receptors and inhibit or activate the expression or action of the receptors. Types of agonist and antagonists include, but are not limited to, polypeptides, proteins, peptides, glycoproteins, glycopeptides, glycolipids, polysaccharides, oligosaccharides, nucleotides, organic molecules, bioorganic molecules, peptidomimetics, pharmacological agents and their metabolites, and transcriptional and translation control sequences.

In one embodiment, antagonists are a soluble form of NFAT activating receptors and soluble polypeptides derived from the extracellular domains of NFAT activating receptors that are capable of binding the NFAT activating receptor. Preferably, the antagonists are peptides selected from the group consisting of amino acids 43 to ISO of SEQ ID NO:2 or antagonist fragments thereof. These antagonistic block the binding of the natural ligand for NFAT activating receptors by binding to the ligand and preventing the ligand from binding to the native receptor.

Preferably, the agonists and antagonists are antibodies that bind specifically to the receptors and influence their biological actions and functions, e.g., to activate or inhibit the production of cytokines and cellular receptors. The antibodies can be polyclonal or monoclonal antibodies but are preferably monoclonal antibodies.

Agonist antibodies are used to prevent or treat diseases characterized by relatively low cytokine and receptor expression compared to non-disease states. Antagonist antibodies are used to prevent or treat diseases characterized by relatively high cytokine and receptor expression compared to non-disease states.

The agonists and antagonists are used for the treatment of various immune diseases, including, but not limited to allergic diseases such as asthma, allergic rhinitis, atopic dermatitis, food hypersensitivity and urticaria; transplantation associated diseases including graft rejection and graft-versus-host-disease; autoimmune or immune-mediated skin diseases including bullous skin diseases, erythema multiforme and contact dermatitis, psoriasis; rheumatoid arthritis, juvenile chronic arthritis; inflammatory bowel disease (i.e., ulcerative colitis, Crohn's disease); systemic lupus erythematosis; spondyloarthropathies; systemic sclerosis (scleroderma); idiopathic inflammatory myopathies (dermatomyositis, polymyositis); Sjogren's syndrome; systemic vasculitis; sarcoidosis; autoimmune hemolytic anemia (immune pancytopenia, paroxysmal nocturnal hemoglobinuria), autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointersritial nephritis); demyelinating diseases of the central and peripheral nervous systems such as multiple sclerosis, idiopathic demyelinatingpolyneuropathy or Guillain-Barre syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory and fibrotic lung diseases such as cystic fibrosis, gluten-sensitive enteropathy, and Whipple's disease; immunologic diseases of the lung such as eosinophilic pneumonia, idiopathic pulmonary fibrosis and hypersensitivity pneumonitis.

### Antibody and Antibody Production

In another aspect, the present invention provides an antibody that binds to the NFAT activating receptors of the present invention and methods for producing such antibody, including antibodies that function as native NFAT activating receptor agonists or antagonists. In one embodiment, the method comprises using isolated NFAT activating receptors or antigenic fragments thereof as an antigen for producing antibodies that bind to the NFAT activating receptors of the present invention in a known protocol for producing antibodies to antigens, including polyclonal and monoclonal antibodies. In another embodiment, the method comprises using host cells that express recombinant NFAT activating receptors as an antigen. In a further embodiment, the method comprises using DNA expression vectors containing the receptor gene to express the receptor as an antigen for producing the antibodies.

Methods for producing antibodies, including polyclonal, monoclonal, monovalent, humanized, human, bispecific, and heteroconjugate antibodies, are well known to skilled artisans.

### Polyclonal Antibodies

Polyclonal antibodies can be produced in a mammal by injecting an immunogen alone or in combination with an adjuvant Typically, the immunogen is injected in the mammal using one or more subcutaneous or intraperitoneal injections. The immunogen may include the polypeptide of interest or a fusion protein comprising the polypeptide and another polypeptide known to be immunogenic in the mammal being immunized. The immunogen may also include cells expressing a recombinant receptor or a DNA expression vector containing the receptor gene. Examples of such immunogenic proteins include, but are not limited to, keyhole limpet hemocyanin, serum albumin, bovine thyroglobulin, and soybean trypsin inhibitor. Examples of adjuvants include, but are not limited to, Freund's complete adjuvant and MPL-TDM adjuvant (monophosphoryl Lipid A, synthetic trehalose dicorynomycolate). The immunization protocol may be selected by one skilled in the art without undue experimentation.

### Monoclonal Antibodies

Monoclonal antibodies can be produced using hybridoma methods such as those described by Kohler and Milstein, Nature, 256:495 (1975). In a hybridoma method, a mouse, hamster, or other appropriate host mammal, is immunized with an immunogen to elicit lymphocytes that produce or are capable of producing antibodies that will specifically bind to the immunogen. Alternatively, the lymphocytes may be immunized in vitro. The immunogen will typically include the polypeptide of interest or a fusion protein containing such polypeptide. Generally, peripheral blood lymphocytes (“PBLs”) cells are used if cells of human origin are desired. Spleen cells or lymph node cells are used if cells of non-human mammalian origin are desired. The lymphocytes are then fused with an immortalized cell line using a suitable fusing agent, e.g., polyethylene glycol, to form a hybridoma cell (Goding, Monoclonal Antibodies: Principles and Practice, pp 59-103 (Academic Press, 1986)). Immortalized cell lines are usually transformed mammalian cells, particularly rodent, bovine, or human myeloma cells. Usually, rat or mouse myeloma cell lines are employed. The hybridoma cells may be cultured in a suitable culture medium that preferably contains one or more substances that inhibit the growth or survival of the unfused immortalized cells. For example, if the parental cells lack the enzyme hypoxanthine guanine phosphoribosyl transferase (HGPRT), the culture medium for the hybridomas typically will include hypoxanthine, aminopterin, and thymidine (HAT medium). The HAT medium prevents the growth of HGPRT deficient cells.

Preferred immortalized cell lines are those that fuse efficiently, support stable high level expression of antibody by the selected antibody producing cells, and are sensitive to a medium such as HAT medium. More preferred immortalized cell lines are murine myeloma lines such as those derived from MOPC-21 and MPC-11 mouse tumors available from the Salk Institute Cell Distribution Center, San Diego, Calif. USA, and SP2/0 or X63-Ag8-653 cells available from the American Type Culture Collection, Rockville, Md. USA. Human myeloma and mouse-human heteromyeloma cell lines also have been described for use in the production of human monoclonal antibodies (Kozbor, J. Immunol. 133:3001 (1984); Brodeur et ah, Monoclonal Antibody Production Techniques and Applications, pp. 51-63 (Marcel Dekker, Inc., New York, 1987)). The mouse myeloma cell line NSO may also be used (European Collection of Cell Cultures, Salisbury, Wiltshire UK). Human myeloma and mouse-human heteromyeloma cell lines, well known in the art, can also be used to produce human monoclonal antibodies.

The culture medium used for culturing hybridoma cells can men be assayed for the presence of monoclonal antibodies directed against the polypeptide of interest. Preferably, the binding specificity of monoclonal antibodies produced by the hybridoma cells is determined by immunoprecipitation or by an in vitro binding assay, e.g., radioimmunoassay (RIA) or enzyme-linked immunoabsorbent assay (ELISA). Such techniques and assays are known in the art The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods. Suitable culture media for this purpose include Dulbecco's Modified Eagle's Medium and RPMI-1640 medium. Alternatively, the hybridoma cells may be grown in vivo as ascites in a mammal.

The monoclonal antibodies secreted by the subclones are isolated or purified from the culture medium or ascites fluid by conventional immunoglobulin purification procedures such as protein A-Sepharose, hydroxylapatite chromatography, gel electrophoresis, dialysis, or affinity chromatography.

The monoclonal antibodies may also be produced by recombinant DNA methods, e.g., those described in U.S. Pat No. 4,816,567. DNA encoding the monoclonal antibodies of the invention can be readily isolated and sequenced using conventional procedures, e.g., by using oligonucleotide probes mat are capable of binding specifically to genes encoding the heavy and light chains of murine antibodies (Innis M. et al. In “PCR Protocols. A Guide to Methods and Applications”, Academic, San Diego, Calif. (1990), Sanger, F. S, et al. Proc. Nat Acad. Sci. 74:5463-5467 (1977)). The hybridoma cells described herein serve as a preferred source of such DNA. Once isolated, the DNA may be placed into expression vectors. The vectors are then transfected into host cells such as simian COS cells, Chinese hamster ovary (CHO) cells, or myeloma cells that do not otherwise produce immunoglobulin protein. The recombinant host cells are used to produce the desired monoclonal antibodies. The DNA also may be modified, for example, by substituting the coding sequence for human heavy and light chain constant domains in place of the homologous murine sequences or by covalently joining the immunoglobulin coding sequence to all or part of the coding sequence for a non-immunoglobulin polypeptide. Such a non-immunoglobulin polypeptide can be substituted for the constant domains of an antibody or can be substituted for the variable domains of one antigen combining site of an antibody to create a chimeric bivalent antibody.

Monovalent antibodies can be produced using the recombinant expression of an immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking. Similarly, in vitro methods can be used for producing monovalent antibodies. Antibody digestion can be used to produce antibody fragments, preferably Fab fragments, using known methods.

Antibodies and antibody fragments can be produced using antibody phage libraries generated using the techniques described in McCafferty, et al., Nature 348:552-554 (1990). Clackson, et al., Nature 352:624-628 (1991) and Marks, et al., J. Mol. Biol. 222:581-597 (1991) describe the isolation of murine and human antibodies, respectively, using phage libraries. Subsequent publications describe the production of high affinity (nM range) human antibodies by chain shuffling (Marks, et al., Bio/Technology 10:779-783 (1992)), as well as combinatorial infection and in vivo recombination as a strategy for constructing very large phage libraries (Waterhouse, et al., Nuc. Acids. Res. 21:2265-2266 (1993)). Thus, these techniques are viable alternatives to traditional monoclonal antibody hybridoma techniques for isolation of monoclonal antibodies. Also, the DNA may be modified, for example, by substituting the coding sequence for human heavy-chain and light-chain constant domains in place of the homologous murine sequences (U.S. Pat No. 4,816,567; Morrison, et al., Proc. Nat. Acad. Sci. USA 81:6851 (1984)), or by covalently joining to the immunoglobulin coding sequence all or part of the coding sequence for a non-immunoglobulin polypeptide. Typically, such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

Antibodies can also be produced using use electrical fusion rather than chemical fusion to form hybridomas. This technique is well established. Instead of fusion, one can also transform a B-cell to make it immortal using, for example, an Epstein Barr Virus, or a transforming gene “Continuously Proliferating Human Cell Lines Synthesizing Antibody of Predetermined Specificity,” Zurawaki, V. R. et al, in “Monoclonal Antibodies,” ed. by Kennett R. H. et al, Plenum Press, N.Y. 1980, pp 19-33.

### Humanized Antibodies

Humanized antibodies can be produced using the method described by Winter in Jones et al., Nature, 321:522-525 (1986); Riechmann et al., Nature, 332:323-327 (1988); and Verhoeyen et al., Science, 239:1534-1536 (1988). Humanization is accomplished by substituting rodent CDRs or CDR sequences for the corresponding sequences of a human antibody. Generally, a humanized antibody has one or more amino acids introduced into it from a source that is non-human. Such “humanized” antibodies are chimeric antibodies wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. Humanized forms of non-human (e.g., murine or bovine) antibodies are chimeric immunoglobulins, immunoglobulin chains, or immunoglobulin fragments such as Fv, Fab, Fab′, F(ab′)₂, or other antigen-binding subsequences of antibodies that contain minimal sequence derived from non-human immunoglobulin. Humanized antibodies include human immunoglobulins (recipient antibody) wherein residues from a complementary determining region (CDR) of the recipient are replaced by residues from a CDR of a non-human species (donor antibody) such as mouse, rat, or rabbit having the desired specificity, affinity, and capacity. Sometimes, Fv framework residues of the human immunoglobulin are replaced by corresponding non-human residues. Humanized antibodies also comprise residues that are found neither in the recipient antibody nor in the imported CDR or framework sequences. In general, humanized antibodies comprise substantially all of at least one and typically two variable domains wherein all or substantially all of the CDR regions correspond to those of a non-human immunoglobulin and all or substantially all of the FR regions are those of a human immunoglobulin consensus sequence. Humanized antibodies optimally comprise at least a portion of an immunoglobulin constant region (Fc), typically that of a human immunoglobulin.

### Human Antibodies

Human antibodies can be produced using various techniques known in the art, e.g., phage display libraries as described in Hoogenboom and Winter, J. Mol. Biol., 227:381 (1991) and Marks et al., J. Mol. Biol., 222:581 (1991). Human monoclonal antibodies can be produced using the techniques described in Cole et al., Monoclonal Antibodies and Cancer Therapy, Alan R. Liss, p. 77 (1985) and Boemer et al., J. Immunol., 147(1):86-95 (1991). Alternatively, transgenic animals, e.g., mice, are available which, upon immunization, can produce a full repertoire of human antibodies in the absence of endogenous immunoglobulin production. Such transgenic mice are available from Abgenix, Inc., Fremont, Calif., and Medarex, Inc., Annandale, N.J. It has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA 90:2551 (1993); Jakobovits et al., Nature 362:255-258 (1993); Bruggermann et al., Year in Immunol. 7:33 (1993); and Duchosal et al. Nature 355:258 (1992). Human antibodies can also be derived from phage-display libraries (Hoogenboom et al., J. Mol. Biol. 227:381 (1991); Marks et al., J. Mol. Biol. 222:581-597 (1991); Vaughan, et al., Nature Biotech 14:309 (1996)).

### Bispecific Antibodies

Bispecific antibodies can be produced by the recombinant co-expression of two immunoglobulin heavy-chain/light-chain pairs wherein the two heavy chains have different specificities. Bispecific antibodies are monoclonal, preferably human or humanized, antibodies that have binding specificities for at least two different antigens. In the present invention, one of the binding specificities is for the NFAT activating receptor and the other is for any other antigen, preferably a cell surface receptor or receptor subunit Because of the random assortment of immunoglobulin heavy and light chains, these hybridomas produce a potential mixture of ten different antibodies. However, only one of these antibodies has the correct bispecific structure. The recovery and purification of the correct molecule is usually accomplished by affinity chromatography.

Antibody variable domains with the desired binding specificities (antibody-antigen combining sites) can be fused to immunoglobulin constant domain sequences. The fusion preferably is with an immunoglobulin heavy chain constant domain comprising at least part of the hinge, CH2, and CH3 regions. Preferably, the first heavy-chain constant region (CHI) containing the site necessary for light-chain binding is present in at least one of the fusions. DNAs encoding the immunoglobulin heavy-chain and, if desired, the immunoglobulin light chain is inserted into separate expression vectors and co-transfected into a suitable host organism. Suitable techniques are shown in for producing bispecific antibodies are described in Suresh et al., Methods in Enzymology, 121:210 (1986).

### Heteroconjugate Antibodies

Heteroconjugate antibodies can be produced known protein fusion methods, e.g., by coupling the amine group of one an antibody to a thiol group on another antibody or other polypeptide. If required, a thiol group can be introduced using known methods. For example, immunotoxins comprising an antibody or antibody fragment and a polypeptide toxin can be produced using a disulfide exchange reaction or by forming a thioether bond. Examples of suitable reagents for this purpose include iminothiolate and methyl-4-mercaptobutyrimidate. Such antibodies can be used to target immune system cells to unwanted cells or to treat HTV infections.

### Polynucleotides

In another aspect, the present invention provides an isolated polynucleotide comprising a nucleotide sequence selected from the group consisting of SEQ ID NO:1; a variant of SEQ ID NO:1; a fragment of SEQ ID NO:1; a nucleotide sequence mat encodes a polypeptide having the amino acid sequence selected from the group consisting of SEQ ID NO:2; a variant of SEQ ID NO:2; and a fragment of SEQ ID NO:2. In one embodiment, the isolated polynucleotide comprises a nucleotide sequence that encodes a polypeptide having an amino acid sequence selected from the group consisting of amino acids 43 to 150 of SEQ ID NO:2 or antagonist fragments thereof.

The isolated polynucleotides of the present invention are preferably coding sequences for NFAT activating receptors involved in the transcriptional regulation of various cytokine and receptor genes. The polynucleotides are used to produce NFAT activating receptors that function as antigens in the process used to produce the agonist and antagonist antibodies that specifically bind to NFAT activating receptors and inhibit or activate the expression or action of such receptors.

### Vectors and Host Cells

In another aspect, the present invention provides a vector comprising a nucleotide sequence encoding the NFAT activating receptors of the present invention and a host cell comprising such a vector.

By way of example, the host cells may be mammalian cells, (e.g. CHO cells), prokaryotic cells (e.g., E. coli) or yeast cells (e.g., Saccharomyces cerevisiae). A process for producing vertebrate fused polypeptides is further provided and comprises culturing host cells under conditions suitable for expression of vertebrate fused and recovering the same from the cell culture.

### Recombinant Expression for NFAT Activating Receptors

Isolated and purified recombinant NFAT activating receptors are provided according to the present invention by incorporating the corresponding nucleotide sequence into expression vectors and expressing the nucleotide sequence in suitable host cells to produce the polypeptide.

### Expression Vectors

Recombinant expression vectors containing a nucleotide sequence encoding the polypeptide can be prepared using well known techniques. The expression vectors include a nucleotide sequence operably linked to suitable transcriptional or translational regulatory nucleotide sequences such as those derived from mammalian, microbial, viral, or insect genes. Examples of regulatory sequences include transcriptional promoters, operators, enhancers, mRNA ribosomal binding sites, and appropriate sequences which control transcription and translation initiation and termination. Nucleotide sequences are “operably linked” when the regulatory sequence functionally relates to the nucleotide sequence for the appropriate polypeptide. Thus, a promoter nucleotide sequence is operably linked to a NFAT activating receptor sequence if the promoter nucleotide sequence controls the transcription of the appropriate nucleotide sequence.

The ability to replicate in the desired host cells, usually conferred by an origin of replication and a selection gene by which transformants are identified, may additionally be incorporated into the expression vector.

In addition, sequences encoding appropriate signal peptides that are not naturally associated with NFAT activating receptors can be incorporated into expression vectors. For example, a nucleotide sequence for a signal peptide (secretory leader) may be fused in-frame to the polypeptide sequence so that the polypeptide is initially translated as a fusion protein comprising the signal peptide. A signal peptide that is functional in the intended host cells enhances extracellular secretion of the appropriate polypeptide. The signal peptide may be cleaved from the polypeptide upon secretion of polypeptide from the cell.

### Host Cells

Suitable host cells for expression of NFAT activating receptors include prokaryotes, yeast, archae, and other eukaryotic cells. Appropriate cloning and expression vectors for use with bacterial, fungal, yeast, and mammalian cellular hosts are well known in the art, e.g., Pouwels et al. Cloning Vectors: A Laboratory Manual, Elsevier, New York (1985). The vector may be a plasmid vector, a single or double-stranded phage vector, or a single or double-stranded RNA or DNA viral vector. Such vectors may be introduced into cells as polynucleotides, preferably DNA, by well known techniques for introducing DNA and RNA into cells. The vectors, in the case of phage and viral vectors also may be and preferably are introduced into cells as packaged or encapsulated virus by well known techniques for infection and transduction. Viral vectors may be replication competent or replication defective. In the latter case viral propagation generally will occur only in complementing host cells. Cell-free translation systems could also be employed to produce the protein using RNAs derived from the present DNA constructs.

Prokaryotes useful as host cells in the present invention include gram negative or gram positive organisms such as E. coli or Bacilli, hi a prokaryotic host cell, a polypeptide may include a N-terminal methionine residue to facilitate expression of the recombinant polypeptide in the prokaryotic host cell. The N-terminal Met may be cleaved from the expressed recombinant NFAT activating receptor polypeptide. Promoter sequences commonly used for recombinant prokaryotic host cell expression vectors include (β-lactamase and the lactose promoter system.

Expression vectors for use in prokaryotic host cells generally comprise one or more phenotypic selectable marker genes. A phenotypic selectable marker gene is, for example, a gene encoding a protein that confers antibiotic resistance or that supplies an autotrophic requirement Examples of useful expression vectors for prokaryotic host cells include those derived from commercially available plasmids such as the cloning vector pBR322 (ATCC 37017). pBR322 contains genes for ampicillin and tetracycline resistance and thus provides simple means for identifying transformed cells. To construct an expression vector using pBR322, an appropriate promoter and a DNA sequence are inserted into the pBR322 vector. Other commercially available vectors include, for example, pKK223-3 (Pharmacia Fine Chemicals, Uppsala, Sweden), pGEM1 (Promega Biotec, Madison, Wis., USA), and the pET (Novagen, Madison, Wis., USA) and pRSET (Invitrogen Corporation, Carlsbad, Calif., USA) series of vectors (Studier, F. W., J. Mol. Biol. 219:37 (1991); Schoepfer, R. Gene 124:83 (1993)).

Promoter sequences commonly used for recombinant prokaryotic host cell expression vectors include T7, (Rosenberg, A. H., Lade, B. N., Chui, D- S., Lin, S- W., Dunn, J. J., and Studier, F. W. (1987) Gene (Amst) 56, 125-135), β-lactamase (penicillinase), lactose promoter system (Chang et al., Nature 275:615, (1978); and Goeddel et al., Nature 281:544, (1979)), tryptophan (tip) promoter system (Goeddel et al., Nucl. Acids Res. 8:4057, (1980)), and tac promoter (Maniatis, Molecular Cloning: A Laboratory Manual, Cold Spring Harbor Laboratory, p. 412 (1982)).

Yeasts useful as host cells in the present invention include those from the genus Saccbaromyces, Pichia, K. Actinomycetes and Kluyveromyces. Yeast vectors will often contain an origin of replication sequence from a 2μ yeast plasmid, an autonomously replicating sequence (ARS), a promoter region, sequences for polyadenylation, sequences for transcription termination, and a selectable marker gene. Suitable promoter sequences for yeast vectors include, among others, promoters for metallothionein, 3-phosphoglycerate kinase (Hitzeman et al., J. Biol. Chem. 255:2073, (1980)) or other glycolytic enzymes (Holland et al., Biochem. 17:4900, (1978)) such as enolase, glyceraldehyde-3-phosphate dehydrogenase, hexokinase, pyruvate decarboxylase, phosphofructokinase, ghicose-6-phosphate isomerase, 3-phosphogfycerate mutase, pyruvate kinase, triosephospbate isomerase, phosphoghicose isomerase, and glucokinase. Other suitable vectors and promoters for use in yeast expression are further described in Fleer et al., Gene, 107:285-195 (1991). Other suitable promoters and vectors for yeast and yeast transformation protocols are well known in the art

Yeast transformation protocols are known to those of skill in the art One such protocol is described by Hinnen et al., Proceedings of the National Academy of Sciences USA, 75:1929 (1978). The Hinnen protocol selects for Trp.sup.+ transformants in a selective medium, wherein the selective medium consists of 0.67% yeast nitrogen base, 0.5% casamino acids, 2% glucose, 10 μg/ml adenine, and 20 μg/ml uracil.

Mammalian or insect host cell culture systems well known in the art could also be employed to express recombinant NFAT activating receptors, e.g., Baculovirus systems for production of heterologous proteins in insect cells (Luckow and Summers, Bio/Technology 6:47 (1988)) or Chinese hamster ovary (CHO) cells for mammalian expression may be used. Transcriptional and translational control sequences for mammalian host cell expression vectors may be excised from viral genomes. Commonly used promoter sequences and enhancer sequences are derived from Polyoma virus, Adenovirus 2, Simian Virus 40 (SV40), and human cytomegalovirus. DNA sequences derived from the SV40 viral genome may be used to provide other genetic elements for expression of a structural gene sequence in a mammalian host cell, e.g., SV40 origin, early and late promoter, enhancer, splice, and polyadenylation sites. Viral early and late promoters are particularly useful because both are easily obtained from a viral genome as a fragment which may also contain a viral origin of replication. Exemplary expression vectors for use in mammalian host cells are well known in the art

NFAT activating receptors may, when beneficial, be expressed as a fusion protein mat has the NFAT activating receptor attached to a fusion segment The fusion segment often aids in protein purification, e.g., by permitting the fusion protein to be isolated and purified by affinity chromatography. Fusion proteins can be produced by culturing a recombinant cell transformed with a fusion nucleic acid sequence that encodes a protein including the fusion segment attached to either the carboxyl and/or amino terminal end of the protein. Preferred fusion segments include, but are not limited to, gtutathione-S-transferase, β-galactosidase, a poly-histidine segment capable of binding to a divalent metal ion, and maltose binding protein.

### Expression and Recovery

According to the present invention, isolated and purified NFAT activating receptors may be produced by the recombinant expression systems described above. The method comprises culturing a host cell transformed with an expression vector comprising a nucleotide sequence that encodes the polypeptide under conditions sufficient to promote expression of the polypeptide. The polypeptide is then recovered from culture medium or cell extracts, depending upon the expression system employed. As is known to the skilled artisan, procedures for purifying a recombinant polypeptide will vary according to such factors as the type of host cells employed and whether or not the recombinant polypeptide is secreted into the culture medium. When expression systems that secrete die recombinant polypeptide are employed, the culture medium first may be concentrated. Following the concentration step, the concentrate can be applied to a purification matrix such as a gel filtration medium. Alternatively, an anion exchange resin can be employed, e.g., a matrix or substrate having pendant diethylaminoethyl (DEAE) groups. The matrices can be acrylamide, agarose, dextran, cellulose, or other types commonly employed in protein purification. Also, a cation exchange step can be employed. Suitable cation exchangers include various insoluble matrices comprising sulfopropyl or carboxymethyl groups. Further, one or more reversed-phase high performance liquid chromatography (RP-HPLQ steps employing hydrophobic RP-HPLC media (e.g., silica gel having pendant methyl or other aliphatic groups), ion exchange-HPLC (e.g., silica gel having pendant DEAE or sulfopropyl (SP) groups), or hydrophobic interaction-HPLC (e.g., silica gel having pendant phenyl, butyl, or other hydrophobic groups) can be employed to further purify the protein. Some or all of the foregoing purification steps, in various combinations, are well known in the art and can be employed to provide an isolated and purified recombinant polypeptide.

Recombinant polypeptide produced in bacterial culture is usually isolated by initial disruption of the host cells, centrifugation, extraction from cell pellets if an insoluble polypeptide, or from the supernatant fluid if a soluble polypeptide, followed by one or more concentration, salting-out, ion exchange, affinity purification, or size exclusion chromatography steps. Finally, RP-HPLC can be employed for final purification steps. Microbial cells can be disrupted by any convenient method, including freeze-thaw cycling, sonication, mechanical disruption, or use of cell tysing agents.

### Agonists and Antagonists Screening

In another aspect, the present invention provides a screening method for identifying NFAT activating receptor agonists and antagonists. The screening method comprises exposing a NFAT activating receptor to a potential NFAT agonist/NFAT antagonist and determining whether the potential agonist/antagonist binds to the receptor. If the potential agonist/antagonist binds to the receptor, there is a strong presumption that the potential agonist/antagonist will actually function as an agonist or antagonist when administered in vivo to a patient and exposed to the native NFAT activating receptor. The NFAT agonists and NFAT antagonists identified using the method can be characterized as an agonist or an antagonist by exposing cells capable of producing cytokines to the agonist/antagonist and measuring cytokine production in comparison to non-exposed cells. Agonists will increase cytokine production; antagonists will decrease cytokine production. Another method for screening comprises transfecting the cells with a reporter gene constructs that contains NFAT DNA binding sequences. Preferably, the potential agonist/antagonist is an organic compound or polypeptide, including antibodies. The screening methods are useful for identifying compounds that may function as drugs for preventing or treating diseases, particularly diseases characterized by relatively low or relatively high cytokine production compared to non-disease states.

### Adverse Side Effect Screening

In a further aspect, the present invention provides a screening method for determining whether pharmaceuticals are likely to cause undesirable side effects associated with reducing or increasing cytokine and cellular receptor production when administered to an animal for the desired indication. The screening method comprises exposing NFAT activating receptors to the pharmaceutical and determining whether the pharmaceutical binds to the receptors or mimics the biological function of the receptor ligand by causing a change in cytokine production. If the pharmaceutical binds to the receptors or mimics the biological function of the receptor ligand, mere is a likelihood that the pharmaceutical will cause adverse side effects when administered to an animal for the desired indication. The adverse side effects result from an undesirable change in cytokine production. Pharmaceuticals mat can be screened by this method include, but are not limited to, polypeptides, proteins, peptides, glycoproteins, glycopeptides, glycolipids, polysaccharides, oligosaccharides, nucleotides, organic molecules, bioorganic molecules, peptidomimetics, pharmacological agents and their metabolites, and transcriptional and translation control sequences. In a preferred embodiment, antibodies to be administered for a particular indication are screened to see if they cross-react with NFAT activating receptors and are therefore likely to cause unwanted side effects when administered for the intended indication.

### Receptor Expression Modulation

In yet another aspect, the present invention provides a method for blocking or modulating the expression of a cellular NFAT activating receptor by interfering with the transcription or translation of a DNA or RNA polynucleotide encoding the NFAT activating receptor. The method comprises exposing a cell capable of expressing a NFAT activating receptor to a molecule that interferes with the proper transcription or translation of a DNA or RNA polynucleotide encoding the NFAT activating receptor. The molecule can be an organic molecule, a bioorganic molecule, an antisense nucleotide, a RNAi nucleotide, or a ribozyme.

In a preferred embodiment, the method comprises blocking or modulating the expression of cellular NFAT activating receptors by exposing a cell to a polynucleotide that is antisense to or forms a triple helix with NFAT activating receptor-encoding DNA or with DNA regulating expression of NFAT activating receptor-encoding DNA. The cell is exposed to antisense polynucleotide or triple helix-forming polynucleotide in an amount sufficient to inhibit or regulate expression of the NFAT activating receptor. Also, the present invention provides a method for blocking or modulating expression of NFAT activating receptors in an animal by administering to the animal a polynucleotide that is antisense to or forms a triple helix with NFAT activating receptor-encoding DNA or with DNA regulating expression of NFAT activating receptor-encoding DNA. The animal is administered antisense polynucleotide or triple helix-forming polynucleotide in an amount sufficient to inhibit or regulate expression of NFAT activating receptor in the animal. Preferably, the antisense polynucleotide or triple helix-forming polynucleotide is a DNA or RNA polynucleotide.

Methods for exposing cells to antisense polynucleotides and for administering antisense polynucleotides to animals are well known in the art In a preferred method, the polynucleotide is incorporated into the cellular genome using know methods and allowed to be expressed inside the cell. The expressed antisense polynucleotide binds to polynucleotides coding for NFAT activating receptors and interferes with their transcription or translation.

The methods are useful for inhibiting cytokine and receptor expression while conducting research on various types of cells, e.g., neutrophils or mast cells, and for preventing or treating animal disease characterized by excess cytokine production compared to non-disease states.

### Disease Predisposition Diagnostic

In another aspect, the present invention provides a method for diagnosing the predisposition of a patient to develop diseases caused by the unregulated expression of cytokines. The invention is based upon the discovery that the presence of or increased amount of NFAT activating receptors in certain patient cells, tissues, or body fluids indicates that the patient is predisposed to certain immune diseases. In one embodiment, the method comprises collecting a cell, tissue, or body fluid sample known to contain few if any NFAT activating receptors from a patient, analyzing the tissue or body fluid for the presence of NFAT activating receptor in the tissue, and predicting the predisposition of the patient to certain immune diseases based upon the presence of NFAT activating receptor in the tissue or body fluid. In another embodiment, the method comprises collecting a cell, tissue, or body fluid sample known to contain a defined level of NFAT activating receptors from a patient, analyzing the tissue or body fluid for the amount of NFAT activating receptor in the tissue, and predicting the predisposition of the patient to certain immune diseases based upon the change in the amount of NFAT activating receptor in the tissue or body fluid compared to a defined or tested level established for normal cell, tissue, or bodly fluid. The defined level of NFAT activating receptor may be a known amount based upon literature values or may be determined in advance by measuring the amount in normal cell, tissue, or body fluids. Specifically, determination of NFAT activating receptor levels in certain tissues or body fluids permits specific and early, preferably before disease occurs, detection of immune diseases in the patient. Immune diseases mat can be diagnosed using the present method include, but are not limited to, the immune diseases described herein. In the preferred embodiment, the tissue or body fluid is peripheral blood, peripheral blood leukocytes, biopsy tissues such as lung or skin biopsies, and synovial fluid and tissue.

### Disease Prevention and Treatment

In another aspect, the present invention provides a method for preventing or treating NFAT protein mediated diseases in a mammal. The method comprises administering a disease preventing or treating amount of a NFAT activating receptor agonist or antagonist to the mammal. The agonist or antagonist binds to the NFAT activating receptor and regulates cytokine and cellular receptor expression to produce cytokine levels characteristic of non-disease states. Preferably, the disease is an allergy, asthma, autoimmune, or other inflammatory disease. Most preferably, the disease is an allergy or asthma.

The dosages of NFAT activating receptor agonist or antagonist vary according to the age, size, and character of the particular mammal and the disease. Skilled artisans can determine the dosages based upon these factors. The agonist or antagonist can be administered in treatment regimes consistent with the disease, e.g., a single or a few doses over a few days to ameliorate a disease state or periodic doses over an extended time to prevent allergy or asthma.

The agonists and antagonists can be administered to the mammal in any acceptable manner including by injection, using an implant, and the like. Injections and implants are preferred because they permit precise control of the timing and dosage levels used for administration. The agonists and antagonists are preferably administered parenterally. As used herein parenteral administration means by intravenous, intramuscular, or intraperitoneal injection, or by subcutaneous implant

When administered by injection, the agonists and antagonists can be administered to the mammal in a injectable formulation containing any biocompatible and agonists and antagonists compatible carrier such as various vehicles, adjuvants, additives, and diluents. Aqueous vehicles such as water having no nonvolatile pyrogens, sterile water, and bacteriostatic water are also suitable to form injectable solutions. In addition to these forms of water, several other aqueous vehicles can be used. These include isotonic injection compositions that can be sterilized such as sodium chloride, Ringer's, dextrose, dextrose and sodium chloride, and lactated Ringer's. Nonaqueous vehicles such as cottonseed oil, sesame oil, or peanut oil and esters such as isopropyl myristate may also be used as solvent systems for the compositions. Additionally, various additives which enhance the stability, sterility, and isotonicity of the composition including antimicrobial preservatives, antioxidants, chelating agents, and buffers can be added. Any vehicle, diluent, or additive used would, however, have to be biocompatible and compatible with the agonists and antagonists according to the present invention.

### NFAT Polypeptide Diagnostic

The antibodies of the present invention may also be used in a diagnostic method for detecting NFAT activating receptors expressed in specific cells, tissues, or body fluids or their components. The method comprises exposing cells, tissues, or body fluids or their components to an antibody of the present invention and determining if the cells, tissues, or body fluids or their components bind to the antibody. Cells, tissues, or body fluids or their components that bind to the antibody cells, tissues, or body fluids or their components that bind to the antibody are diagnosed as cells, tissues, or body fluids that contain NFAT activating receptors. Such method is useful for determining if a particular cell, tissue, or body fluid is a one of a certain type of cell, tissue, or body fluid previously known to contain NFAT activating receptors. Various diagnostic methods known in the art may be used, e.g., competitive binding assays, direct or indirect sandwich assays, and immunoprecipitation assays conducted in either heterogeneous or homogeneous phases.

### NFAT Polypeptide Purification

The antibodies of the present invention may also be used in a method for isolating and purifying NFAT activating receptors from recombinant cell cultures, contaminants, and native environments. The method comprises exposing a composition containing NFAT activating receptors and contaminants to an antibody capable of binding to the receptors, allowing the NFAT activating receptors to bind to the antibody, separating the antibody-receptor complexes from the contaminants, and recovering the NFAT activating receptors from the complexes. Various purification methods known in the art may be used, e.g., affinity purification methods that recover NFAT activating receptors from recombinant cell culture or native sources. In this method, the antibodies against NFAT activating receptors are immobilized on a suitable support such a Sephadex resin or filter paper using methods well known in the art. The immobilized antibody then is contacted with a sample composition containing the NFAT activating receptors to be purified and contaminants. The support is then washed with a suitable solvent capable of removing substantially all the material in the sample except the NFAT activating receptors bound to the immobilized antibody. Finally, the support is washed with another suitable solvent that that removes the NFAT activating receptors from the antibody.

### Tolerance Induction Method

In another aspect, the present invention provides a method for inducing tolerance in a mammal that may experience an unwanted immune response. The method comprises administering a NFAT activating receptor antagonist to the patient in amounts sufficient to inhibit the translocation of NFAT protein into the cell nucleus and its subsequent interaction with the transcription factor activator protein 1 (AP-1), a transcription factor known to interact with NFAT protein. Preferably, the antagonist is an antibody that binds to a NFAT activating receptor and prevents NFAT proteins from translocating to the nucleus and interacting with AP-1.

Tolerance is induced through a process of incomplete signaling. Self-antigens stimulate only the T cell receptor and cause an increase in intracellular calcium levels that activate NFAT proteins. NFAT proteins then bind to specific sites of T cell DNA and trigger gene expression that induces tolerance. However, NFAT proteins typically interact with another transcription factor known as AP-1. The interaction between these transcription factors induce a full immune response wherein T cells fight foreign antigens. However, if NFAT proteins do not interact with AP-1, a state of T cell unresponsiveness wherein the T cells tolerate antigens is produced. A full immune response to foreign antigens is the cause of many unwanted immune responses, e.g., allergies and transplant organ rejections. Therefore, any agent that prevents the interaction of NFAT and AP-1 will prevent these unwanted and destructive responses and induce tolerance.

In a preferred embodiment, the present invention provides a method for inducing tolerance in an organ transplant patient Generally, a full immune response to a transplanted organ's foreign antigens is the cause of organ rejection. Currently, the immunosuppressive drug cyclosporin is used by transplant patients to prevent rejection by shutting down the activity of NFAT proteins. However, this use of cyclosporin prevents NFAT protein initiation of T cell tolerance. Therefore, preventing the interaction of NFAT protein and AP-1 will induce tolerance to the transplanted organ.

### Knockout Animals

In another aspect, the present invention provides a knockout animal comprising a genome having a heterozygous or homozygous disruption in its endogenous NFAT activating receptor gene that suppresses or prevents the expression of biologically functional NFAT activating receptor proteins. Preferably, the knockout animal of the present invention has a homozygous disruption in its endogenous NFAT activating receptor gene. Preferably, the knockout animal of the present invention is a mouse. The knockout animal can be made easily using techniques known to skilled artisans. Gene disruption can be accomplished in several ways including introduction of a stop codon into any part of the polypeptide coding sequence that results in a biologically inactive polypeptide, introduction of a mutation into a promoter or other regulatory sequence that suppresses or prevents polypeptide expression, insertion of an exogenous sequence into the gene that inactivates the gene, and deletion of sequences from the gene.

Several techniques are available to introduce specific DNA sequences into the mammalian germ line and to achieve stable transmission of these sequences (transgenes) to each subsequent generation. The most commonly used technique is direct microinjection of DNA into the pronucleus of fertilized oocytes. Mice or other animals derived from these oocytes will be, at a frequency of about 10 to 20%, the transgenic founders mat through breeding will give rise to the different transgenic mouse lines. Methods for generating transgenic animals via embryo manipulation and microinjection, particularly animals such as mice, have become conventional in the art, e.g., U.S. Pat. Nos. 4,736,866, 4,870,009, and 4,873,191 and in Hogan, B., Manipulating the Mouse Embryo, (Cold Spring Harbor Laboratory Press, Cold Spring Harbor, N.Y., 1986). Similar methods are used for production of other transgenic animals.

Embryonic stem cell (“ES cell”) technology can be used to create knockout mice (and other animals) with specifically deleted genes. Totipotent embryonic stem cells, which can be cultured in vitro and genetically modified, are aggregated with or microinjected into mouse embryos to produce a chimeric mouse that can transmit this genetic modification to its offspring. Through directed breeding, a mouse can thus be obtained that lacks this gene. Several other methods are available for the production of genetically modified animals, e.g., the intracytoplasmic sperm injection technique (ICSI) can be used for transgenic mouse production. This method requires microinjecting the head of a spermatocyte into the cytoplasm of an unfertilized oocyte, provoking fertilization of the oocyte, and subsequent activation of the appropriate cellular divisions of a preimplantation embryo. The mouse embryos thus obtained are transferred to a pseudopregnant receptor female. The female will give birth to a litter of mice. In ICSI applied to transgenic mouse production, a sperm or spermatocyte heads suspension is incubated with a solution containing the desired DNA molecules (transgene). These interact with the sperm that, once microinjected, act as a carrier vehicle for the foreign DNA. Once inside the oocyte, the DNA is integrated into the genome, giving rise to a transgenic mouse. This method renders higher yields (above 80%) of transgenic mice than those obtained to date using traditional pronuclear microinjection protocols.

### EXAMPLES

This invention can be further illustrated by the following examples of preferred embodiments thereof, although it will be understood that these examples are included merely for purposes of illustration and are not intended to limit the scope of the invention unless otherwise specifically indicated.

### Example 1

### Identification of NFAT Activating Receptor Gene

Non-redundant human protein database IPI (International Protein Index) was searched for novel molecules containing: 1) immunoglobulin (Ig) domain, 2) immunoreceptor tyrosine-based activation motif (ITAM), and 3) transmembrane region. Those are common features shared by many signal activating receptors mediating immune system functions, including components of TCR, BCR, FceRI and many other recently identified activating receptors (Isakov 1997 “ITIMs and ITAMs” Immunologic Research 16:85). A Hidden Markov Model (HMM)-based method was employed for Ig-domam search. The HMM, which was built from an alignment of 113 confident Ig domains and calibrated using program HMMER, was obtained from Pfam (version 6.6) database. To search proteins containing ITAM motif, a PROSITE-formatted motif profile was first constructed based on the common features of ITAM motif, and software “seedtop” (NCBI) was used to do the search. Large-scale transmembrane region prediction for all the IPI proteins was carried out by using software TMHMM version 2.0 (http://www.cbs.dm.dk/sevices/TMHMM/). A hypothetical protein sequence labeled as “IPI00086590” was found to meet all the three criteria. In silico cloning procedure was used to derive its full- length cDNA sequence shown in SEQ ID NO:2.

### Example 2

### Quantitative Real-time PCR Analysis of NFAT Activating Receptor Gene Expression

Oligonucleotide primers having the sequence Forward: 5′ TCCTGCTCTTTGGCTTCACC and Reverse: 5′ GCCGTGCCCACTACACTCA were selected from the NFAT activating receptor nucleotide sequences using Primer Express 2.0 (Applied Biosystems, Inc.) and were synthesized and used in PCR reactions to monitor the expression of NFAT activating receptor polynucleotide real-time. RNAs were isolated to measure the level of expression of NFAT activating receptor polynucleotide in the following tissues and cells: brain; heart; kidney; liver; lung, spleen, monocytes, Daudi, a Burkitt's lymphoma cell line; HPB-ALL, a T cell leukemia cell line; THP-1, acute monocytic leukemia; lymphocytes; Jurkat, a T cell leukemia cell line; HMC-1; a mast cell line; HUVAC; primary human vascular endothelial cells; neutrophils; PBMC, peripheral blood mononuclear cells; and four different batches of in vitro cultured cord-blood derived mast cell samples.

Real-time quantitative PCR analysis was performed with the ABI Prism 7900 (Applied Biosystems, Inc.) sequence detection system, using Taqman reagents, according to the manufacture's instructions. Equal amounts of first strand cDNA from the cell sources indicated above were used as PCR templates in reactions to obtain the threshold cycle (Cₜ), and the Cₜ was normalized using the known Cₜ from 18S RNAs to obtain ΔCₜ. To compare relative levels of gene expression of NFAT activating receptor polynucleotide in different cell lines, ΔΔCₜ values were calculated by using the lowest expression level as the base, which were men converted to real fold expression difference values. The results are shown in Table 1.

TABLE 1

| | Relative Expression |
|---|---|
| Tissue/Cell/Cell Line Set 1 | |
| Brain | 530 |
| Heart | 432 |
| Kidney | 258 |
| Liver | 191 |
| Lung | 1411 |
| Spleen | 4108 |
| Monocytes | 19550 |
| Daudi | 443 |
| HPB-ALL | 3 |
| THP-1 | 18441 |
| Lymphocytes | 28570 |
| Jurkat | 4 |
| Cultured mast cells | 5082 |
| HMC-1 | 344 |
| HUVEC | 1 |
| Cell/Cell Line Set 2 | |
| HMC-1 | 1 |
| Daudi | 3 |
| THP-1 | 81 |
| Neutrophils | 362 |
| PBMC | 79 |
| Cultured mast cells (batch 1, week 4) | 23 |
| Cultured mast cells (batch 1, week 7) | 56 |
| Cultured mast cells (batch 2, week 7) | 84 |

Referring to Table 1, the data show that NFAT activating receptor mRNA was found to be highly expressed in neutrophils, primary monocytes and monocytic cell lines, lymphocytes and in vitro cultured mast cells. Expression was detected in spleen and hing tissues. In contrast, the level of expression in all other tissues and cells (brain, heart; kidney, liver, Daudi, HPB-ALL, Jurkat, HUVAC) was very low.

### Example 3

### Molecular Cloning and Characterization of NFAT Activating Receptor

Two oligo primers encompassing the starting methionine codon (5′-CACCATG GAGAACCAGCCTG) and stop codon (5′-ACCTGGTCTATGAAAATCTQ respectively were used to clone NFAT activating receptor cDNA from human monocytes by RT-PCR. Two cDNA clones were isolated, sequenced, and found to be identical to the in-silico cloning-derived coding region sequence shown in SEQ ED NO:1. The in-silico cloning-derived sequence likely represents the complete coding region of NFAT activating receptor polynucleotide as it contains a perfect Kozak motif, and has several in-frame stop codons preceding the predicted initiation methionine. Furthermore, ft has a putative signal peptide starting from the assumed initiation methionine.

NFAT activating receptor was predicted to be a type I transmembrane protein of 270 amino acids with a calculated molecular mass of approximately 30 kD, a putative signal peptide at the N-terminal (amino acids 1-42), an Ig-domain (amino acids 43-150) in the extracellular region, a transmembrane domain (amino acids 164-186) and an IT AM motif (amino acids 220-235) in the cytoplasmic region. It is located at chromosome 22q13.2. Alignment of cDNA with genomic sequence showed mat the coding region of NFAT activating receptor polynucleotide comprises six exons. One potential N-grycosylation site was found in the extracellular region (amino acids 107-110). “Electronic northern” based on the distribution of corresponding EST library sources indicated that NFAT activating receptor is preferentially expressed in leukocytes. By using a web-based SCANSITE, which was designed to search for motifs within proteins that are likely to be phosphorylated by specific protein kinases or bind to domains (http://scansite.mit.edu/), NFAT activating receptor was predicted to contain a binding site in the cytoplasmic region for SH2-domain of Lck (lymphocyte—specific protein tyrosine kinase), a very common activation adaptor molecule in signaling transduction.

### Example 4

### Expression of NFAT Activating Receptor

To determine the NFAT activating receptor gene product, the coding region of the polynucleotide that expresses NFAT activating receptor was subcloned into a pcDNA 3.1 mammalian expression vector (INVTTROGEN, CA), with a V5 tag fused in frame to the C-terminus or N-terminus, and then transiently transfected into 293T cells with Lipofectamine 2000. The whole cell protein sample was prepared by re-suspending 8×10⁵ cells in 100 μl of ddH₂O, and heated at 98° C. for 5 minutes after adding equal volume of 2× sample loading buffer. The proteins were separated in a 15% SDS-PAGE and transferred to membrane. The tagged NFAT activating receptor is detected as an approximately 35 kD predominant protein band and an approximately 30 kD minor band by Western blot with anti-V5 monoclonal antibody. These protein bands were not present in the cells transfected with plasmid vector-only.

### Example 5

### Cellular Localization of NFAT Activating Receptor

To determine whether NFAT activating receptor is expressed on the membrane surface, 293T cells were transfected with NFAT activating receptor construct with a V5 tag at its C-terminus, and lysed the cells by freeze-thaw method. Cells were suspended in 1× lysis buffer and freeze-thawed three times. Insoluble membrane fraction was separated from soluble proteins by centrifugation at maximum speed in a microcentrifuge. The proteins were separated in a 15% SDS-PAGE. NFAT activating receptor was present mainly in the membrane fraction as detected by anti-V5 monoclonal antibodies. Little was detected in soluble fraction.

Immunofluorescence staining was then performed to determine the location and orientation of NFAT activating receptor in the membrane. NFAT activating receptor was fused with V5 tag at either N-terminus or C-terminus and transfected into 293T cells. The cells were washed and pre-incubated at 4° C. for 30 minutes in the enzyme-free cell dissociation buffer (Invitrogen) containing 1% BSA. Cells were then incubated with FTTC-conjugated Anti-V5 monoclonal antibodies (10 μg/ml) (Invitrogen) in the same buffer for 30 minutes. After three washes, cells were re-suspended in 1× PBS with/without fixation by 1% paraformaldehyde and analyzed by fluorescent microscopy (ZEISS Axioskop, Germany). It was found that N-terminus tagged NFAT activating receptor was detected by anti-V5 monoclonal antibodies on the membrane of both living (unfixed) and methanol-fixed cells, while C-terminus tagged NFAT activating receptor was detected only on the membrane of methanol-fixed cells. These results show mat NFAT activating receptor is a transmembrane protein with the N-terminus exposed to the outside of the cellular membrane.

### Example 6

### NFAT Activation by NFAT Activating Receptor

It has been known that the first level regulation of activation or inhibition of an immune response occurs at the receptor she and involves protein modules at the cytoplasmic region of receptor subunits. Recent studies led to the identification of two types of modules, IT AM (immunoreceptor tyrosine-based activation motif) and ITIM (immunoreceptor tyrosine-based inhibition motif). They possess conserved tyrosine residues that undergo rapid, but transient phosphorylation upon receptor ligation, and activate or terminate signal transduction pathways (Isakov 1998 “ITAMs: immunoregulatory scaffolds that link immunoreceptors to their intracellular signaling pathways” Receptors Channels 5:243). Immunoglobulin (Ig) domain has been frequently found to be involved in ligand-receptor interaction. The co-existence of Ig domain and IT AM motif in the membrane protein NFAT activating receptor, along with the preferential expression in immune tissues and cells, strongly suggest that NFAT activating receptor functions as an activating receptor in immune system.

Three common transcription factors (NFAT, NF-κB and AP-1) have been tested as potential downstream targets of NFAT activating receptor signaling pathway by luciferase reporter assay in HMC-1 (a human mast cell line), in which NFAT activating receptor was found moderately expressed by real-time PCR analysis. A typical luciferase assay was carried out as follows: HMC-1 was seeded in a 24-well culture plate at the density of 0.2 million cells per milliliter of medium. Three plasmids, luciferase reporter (with promoter region containing NFAT, AP-1 or NF-κB binding site), NFAT activating receptor expression plasmid and pRL-SV40, were co-transfected in HMC-1 cells. The cells were harvested 40-46 hours after transfection and lysed in passive lysis buffer (Promega, Inc.). The firefly and Renila luciferase activities were assayed with the dual luciferase assay kit (Promega, Inc.) and by TD-20 luminometer (Turner Design). The results are shown in Tables 2 through 7.

TABLE 2
NFAT - Luciferase Reporter Assay

| Plasmid | Relative Luciferase Activity |
|---|---|
| Vector-only | 1.0 |
| V5-353 | 79.8 |
| V5-353Y1A | 0.6 |
| V5-353Y2A | 1.6 |
| V5-353Y12A | 0.8 |

TABLE 3
NF-κB - Luciferase Reporter Assay

| Plasmid | Relative Luciferase Activity |
|---|---|
| Vector-only | 1.0 |
| V5-353 | 1.0 |
| V5-353Y1A | 0.7 |
| V5-353Y2A | 0.3 |
| V5-353Y12A | 0.3 |
| MEKK3 | 233.0 |

TABLE 4
AP-1 - Luciferase Reporter Assay

| Plasmid | Relative Luciferase Activity |
|---|---|
| Vector-only | 1.0 |
| V5-353 | 1.5 |
| V5-353Y1A | 1.1 |
| V5-353Y2A | 1.6 |
| V5-353Y12A | 1.4 |
| MEKK3 | 200.0 |

TABLE 5
IL-13 - Luciferase Reporter Assay

| Plasmid | Relative Luciferase Activity |
|---|---|
| Vector-only | 1.0 |
| V5-353 | 19.2 |
| V5-353Y1A | 1.3 |
| V5-353Y2A | 1.5 |
| V5-353Y12A | 1.3 |

TABLE 6
TNF-α - Luciferase Reporter Assay

| Plasmid | Relative Luciferase Activity |
|---|---|
| Vector-only | 1.0 |
| V5-353 | 5.1 |
| V5-353Y1A | 1.3 |
| V5-353Y2A | 1.5 |
| V5-353Y12A | 12 |

TABLE 7
Inhibition of NFAT by CsA - Luciferase Reporter Assay

| Concentration of Inhibitor CsA (uM) | Relative Luciferase Activity |
|---|---|
| 0.0 | 31.7 |
| 0.5 | 8.4 |
| 1.0 | 6.4 |
| 2.0 | 7.5 |
| 4.0 | 5.5 |
| 8.0 | 4.8 |

Referring to Tables 2 through 7, the data show that the over-expression of NFAT activating receptor-V5-353 activated NFAT by approximately 80 fold (Table 2), but not NF-κB (Table 3) or AP-1 (Table 4). Vector-only transfection was used as negative control; the negative control did not activate any of the three transcription factors (Tables 2, 3, and 4). In the cases of NF-κB and AP-1, MEKK3 was used as positive control, which is known to be able to activate NF-κB and AP-1 (Tables 3 and 4).

To confirm mat the isolated polypeptide is an NFAT activation receptor, two additional experiments were performed: 1) the effect of over-expression of NFAT activating receptor on the transcription of NFAT regulated genes (IL-13 and TNF-α), and 2) the effect of a known inhibitor of calcinurin/NFAT signaling pathway on NFAT activating receptor mediated NFAT activation. IL-13 and TNF-α were selected because of their pivotal roles in immune responses. The promoter region (containing NFAT binding site) of either IL13 or TNF-α was inserted into the promoterless luciferase reporter plasmid vector (DB Bioscience Clontech), which was then co-transfected into HMC-1. Over-expression of NFAT activating receptor was found to strongly up-regulate the transcription of IL-13 and TNF-a by approximately 19 and 5 fold, respectively, as compared with the vector-only controls (Tables 5 and 6). Furthermore, a known NFAT inhibitor, cyclosporin A (CsA), dramatically reduced NFAT activating receptor's stimulating activity (Table 7). Taken together, these data indicate that overexpression of NFAT activating receptor can activate NFAT.

### Example 7

NFAT Activation by NFAT Activating Receptor is Mediated by ITAM Motif

Structure-function analysis of different receptor subunits has led to the identification of ITAMs in cytoplasmic tails of different antigen and Fc receptors. These receptors operate in a tyrosine phosphorylation-dependent manner and utilize Syk/ZAP-70 PTKs to transduce activation signals (Cambier 1995 “Antigen and Fc receptor signaling” J Immunol 155:3281). The NFAT activating receptor of the present invention was shown to act as an activating receptor with a predicted ITAM motif in the cytoplasmic tails (amino acids 220-235). To determine whether the putative ITAM motif mediates the signal transduction, we generated three NFAT activating receptor mutants (Y1A, Y2A and double mutant Y12A) by mutating the two tyrosines in the ITAM motif (Y220 and Y231, designed Y1 and Y2, respectively) to an alanine individually, as well as in combination. All the mutations were generated by PCR-directed mutagenesis. The primer sequences used were as follows:

| | |
|---|---|
| Y1A | forward: 5′-AGAATCTGTCGCCACAGCTCTG |
| | reverse: 5′-GCAGAGCTGTGGCGACAGATTCTG |
| Y2A | forward: -AGACCGAGGTCGCTGCCTGCATCG |
| | reverse: 5′-CGATGCAGGCAGCGACCTCGGTC |

Mutant cDNAs were then individually subcloned into the pCDNA expression vector (Invitrogen). Similar luciferase reporter assays were then carried out to determine the effect of over-expression of each tyrosine to alanine mutant on the activation of NFAT and the transcription of NFAT-regulated genes (IL-13 and TNF-α). It was found that the NFAT activation activity of each of the three mutants was virtually abolished, the obtained signal of luciferase reporter was comparable to vector-only control (Tables 2,4 and 5). The results show that the activation signaling of NFAT activating receptor is via IT AM motif.

In the specification, there have been disclosed typical preferred embodiments of the invention and, although specific terms are employed, they are used in a generic and descriptive sense only and not for purposes of limitation, the scope of the invention being set forth in the following claims. Obviously many modifications and variations of the present invention are possible in light of the above teachings. It is therefore to be understood that within the scope of the appended claims the invention may be practiced otherwise than as specifically described.

1-31. (canceled)

32. A method for preventing or treating a disease mediated by an NFAT protein in a mammal, the method comprising administering to the mammal an amount of an NFAT activating receptor agonist or antagonist effective for said preventing or treating.

33. The method of claim 32, wherein the disease is an immune disease.

34. The method of claim 33, wherein the disease is selected from the group comprising psoriasis, rheumatoid arthritis, systemic lupus erythematosis, and inflammatory bowel disease.

35. A method for detecting whether an NFAT activating receptor is present in specific cells, tissues, or body fluids, the method comprising:
(a) contacting a sample of cells, tissue, or body fluid with an antibody that binds to the NFAT activating receptor; and
(b) determining whether the antibody binds to NFAT activating receptor in the sample;
wherein the NFAT activating receptor is selected from the group consisting of:
(a) a polypeptide comprising the amino acid sequence of SEQ ID NO: 2;
(b) a polypeptide comprising an amino acid sequence having at least 95% sequence identity to SEQ ID NO: 2; and
(c) a soluble polypeptide comprising or consisting of amino acids 43 to 150 of SEQ ID NO: 2; and
wherein said binding indicates that the NFAT activating receptor is present in said specific cells, tissues, or body fluids.

36. The method of claim 35, wherein the antibody is a polyclonal, monoclonal, humanized, human, bispecific, or heteroconjugate antibody.

37. The method of claim 35, wherein the presence of said NFAT activating receptor indicates a predisposition to develop an immune disease in the subject from which the sample was collected.

38. The method of claim 35, wherein step (b) further determines the amount of the NFAT activating receptor present in the sample.

39. The method of claim 38, wherein the amount of the NFAT activating receptor present in the sample is compared to a defined or tested level established for normal cells, tissues, or body fluids.

40. The method of claim 39, wherein a change in the amount of the NFAT activating receptor compared to the defined or tested level established for normal cells, tissues, or body fluids is indicative of a predisposition to develop an immune disease.

41. The method of claim 40, wherein the disease is caused by the unregulated expression of cytokines.

42. The method of claim 40, wherein the disease is selected from the group comprising psoriasis, rheumatoid arthritis, systemic lupus erythematosis, and inflammatory bowel disease.

43. A purified polypeptide selected from the group consisting of:
a. a polypeptide comprising the amino acid sequence of SEQ ID NO: 2;
b. a polypeptide comprising an amino acid sequence having at least 95% sequence identity to SEQ ID NO: 2;
c. a soluble polypeptide comprising or consisting of amino acids 43 to 150 of SEQ ID NO: 2; and
d. a polypeptide encoded by a polynucleotide comprising the nucleotide sequence of SEQ ID NO: 1.

44. The purified polypeptide of claim 43, wherein the polypeptide is a soluble polypeptide comprising or consisting of amino acids 43 to 150 of SEQ ID NO: 2.

45. An isolated polynucleotide comprising a nucleotide sequence selected from the group consisting of:
a. SEQ ID NO: 1;
b. a nucleotide sequence having at least 85% sequence identity to SEQ ID NO: 1; and
c. a nucleotide sequence that encodes a polypeptide selected from the group consisting of:
i. a polypeptide comprising the amino acid sequence of SEQ ID NO: 2; and
ii. a soluble polypeptide comprising or consisting of amino acids 43-150 of SEQ ID NO: 2.

46. The isolated polynucleotide of claim 45, wherein the polynucleotide comprises a nucleotide sequence that encodes a soluble polypeptide comprising or consisting of amino acids 43-150 of SEQ ID NO: 2.

47. An expression vector comprising the polynucleotide of claim 45.

48. A host cell comprising the polynucleotide of claim 45.

49. A host cell comprising the expression vector of claim 47.

50. A fusion protein comprising the polypeptide of claim 43.

51. A method for producing a purified polypeptide selected from the group consisting of:
a. a polypeptide comprising the amino acid sequence of SEQ ID NO: 2;
b. a polypeptide comprising an amino acid sequence having at least 95% sequence identity to SEQ ID NO: 2;
c. a soluble polypeptide comprising or consisting of amino acids 43 to 150 of SEQ ID NO: 2; and
d. a polypeptide encoded by a polynucleotide comprising the nucleotide sequence of SEQ ID NO: 1,
the method comprising culturing a host cell comprising (i) the polynucleotide of claim 45 or (ii) a host cell comprising an expression vector comprising the polynucleotide of claim 45, under conditions suitable for the expression of the polypeptide and isolating the polypeptide from the host cell.
